(19) United States
(12) Patent Application Publication
(10) Pub. No.: US 2005/0202093 A1
(43) Pub. Date: Sep. 15, 2005

(54) Prolonged suppression of electrical activity in excitable tissues

(76) Inventors: Daniel S. Kohane, Newton, MA (US); Robert S. Langer, Newton, MA (US)

Correspondence Address: CHOATE, HALL & STEWART LLP, EXCHANGE PLACE, 53 STATE STREET, BOSTON, MA, 02109, US

(21) Appl. No.: 10/727,032

(22) Filed: Dec. 2, 2003

Related U.S. Application Data
(60) Provisional application No. 60/430,240, filed on Dec. 2, 2002.

(51) Int. Cl. A61K 9/14; A61K 9/127

(52) U.S. Cl. 424/489; 424/450

(57) ABSTRACT

Controlled release of pharmaceutical agents using microspheres or other controlled release systems are used to treat disease state characterized by aberrant electrical activity in excitable tissue. For the treatment of epilepsy, agents useful in the treatment of epilepsy may be delivered to the patient at the site of seizure origin to control seizure activity in a time release manner. The inventive system may also be useful in the treatment of cardiac arrhythmias and pre-term labor. Particularly useful pharmaceutical compositions comprising a site 1 sodium channel blocker are also provided.

### RELATED APPLICATIONS

The present application claims priority to provisional application, U.S. Ser. No. 60/430,240, filed Dec. 2, 2002, entitled “Prolonged Suppression of Electrical Activity in Excitable Tissues,” which is incorporated herein by reference.

### GOVERNMENT SUPPORT

The work described herein was supported, in part, by grants from the National Institutes of Health (GM00684 and GM26698). The United States government may have certain rights in the invention.

### BACKGROUND OF THE INVENTION

Numerous disease states involve deleterious electrical activity in excitable tissues. These include epilepsy (i.e., aberrant electrical activity in the brain), cardiac arrhythmias (i.e., aberrant electrical activity in the heart or its conduction system), and pre-term labor (i.e., aberrant electrical activity in the uterus). In many cases, sustained attenuation or ablation of the undesired electrical activity would have beneficial effects. Automated delivery systems as well as relatively large implants impregnated with various drugs have been used to deliver pharmacological agents to a specific site of aberrant electrical activity in order to decrease or eliminate the unwanted activity leading to the disease state.

The delivery of a drug to a patient with controlled-release of the active ingredient has been an active area of research for decades and has been fueled by the many recent developments in polymer science and the need to deliver pharmaceutical agents. Biodegradable particles have been developed as sustained release vehicles used in the administration of drugs (Langer Science 249:1527-1533, 1990; Mulligan Science 260:926-932, 1993; Eldridge Mol. Immunol. 28:287-294, 1991; each of which is incorporated herein by reference). The drugs are typically encapsulated in a polymer matrix which is biodegradable and biocompatible. As the polymer is degraded and/or as the drug diffuses out of the polymer, the drug is released into the body. These particles depending on their size, composition, and the drug being delivered can be administered to an individual using any route available.

For example, the treatment of chronic seizure disorders today includes oral pharmacology. Antiepileptic drugs are typically administered multiple times daily. The dosage regimen is determined by the pharmacokinetics of the drug(s) being delivered and their side effects (Cloyd et al. “Antiepileptic drug pharmacokinetics and interactions: impact on treatment of epilepsy” Pharmacotherapy 2000; 20: 139S-151S; French et al. “Antiepileptic drug interactions” Epilepsia 2000; 41: S30-S36; each of which is included herein by reference). Unfortunately, the dose of systemically delivered drug required to achieve a brain concentration sufficient to control seizures may result in unacceptable side effects (Perucca et al. “Harnessing the clinical potential of antiepileptic drug therapy: dosage optimization” CNS Drugs 2001; 15: 609-621; Swann “Major system toxicities and side effects of anticonvulsants” J. Clin. Psychiatry 2001; 62: 16-21; incorporated herein by reference). This is particularly true in some forms of epilepsy (e.g., epilepsia partialis continua), in which seizure activity can be unrelenting. The sequelae of the disorder and the treatments including barbiturate coma and neurosurgery can be severe. A drug delivery system that could directly target the epileptic region in the brain would offer enormous advantages, especially since approximately 60% of seizures are are partial in nature (Hauser et al. “The epidemiology of epilepsy in Rochester, Minn. 1935 through 1967” Epilepsia 1975; 16: 1-66; Hauser et al. “Epilepsy: Frequency, causes and consequences” New York: Demos, 1990; each of which is incorporated herein by reference). Furthermore, status epilepticus is most likely to occur in patients with partial seizures (Hauser “Status epilepticus: epidemiologic considerations” Neurology 1990; 40: 9-13; incorporated herein by reference).

The effectiveness of focally delivered anticonvulsants in treating experimental models of epilepsy has been demonstrated (Eder et al “Local perfusion of diazepam attenuates injterictal and ictal events in the bicuculline model of epilepsy in rats” Epilepsia 1997; 38: 516-521; incorporated herein by reference). Automated systems using a catheter at the epileptogenic focus have been devised that are effective in terminating induced seizures (Stein et al. “An automated drug delivery system for focal epilepsy” Epilepsy Res. 2000; 39: 103-114; incorporated herein by reference). Also, relatively large implants impregnated with various agents have also been found to be effective in animal models (Graber et al. “Tetrodotoxin prevents posttraumatic epileptogenesis in rats” Ann. Neurol. 1999; 46: 234-242; Kubek et al. “Prolonged seizure suppression by a single implantable polymeric-TRH microdisk preparation” Brain Res. 1998; 809: 189-197; Tamargo et al. “The intracerebral administration of phenytoin using controlled-release polymers reduces experimental seizures in rats” Epilepsy Res 2002; 48: 145-155; each of which is incorporated herein by reference).

What is needed is a drug delivery vehicle that will provide prolonged delivery of an agent to a target electrically excitable tissue such as brain to control epilepsy, heart to control arrhythmias, and uterus to control pre-term labor in the form of particles .

### SUMMARY OF THE INVENTION

The present invention provides a drug delivery system for delivering an agent to an electrically excitable tissue in the treatment or prevention of disease states involving aberrant electrical activity. The inventive system includes compositions useful in delivering agents to electrically excitable tissues and methods of treatment using such compositions.

In one embodiment, the drug delivery system includes microparticles containing pharmaceutical agents effective in eliminating or decreasing the unwanted electrical activity in the target organ or a specific area of the target organ. The microparticles are preferably biocompatible and biodegradable and lead to the slow release of the pharmaceutical agent from the microparticles. Typically the size of these particles ranges from 100 micrometers to 50 nanometers. In certain embodiments, the microparticles are lipid-protein-sugar particles; however, as would be appreciated by others of skill in this art, other types of microparticles such as PLGA microspheres may be used in the present invention. Pharmaceutical agents encapsulated in the microparticles include agents that block or inhibit electrical activity in the target organ including anesthetics, anticonvulsants, psychotropic agents, receptor antagonists, ion channel blockers, receptor agonists, etc. These agents may be small molecules, peptides, proteins, and salts. In certain embodiments, the pharmaceutical agent is a combination of a site 1 sodium channel blocker, a local anesthetic, and a glucocorticoid receptor agonist (e.g. dexamethasone).

In one aspect, the inventive system is used to treat epilepsy in a patient suffering therefrom. A person diagnosed with epilepsy may have the site of aberrant electrical activity localized to a particular area of the brain. Microparticles laden with anti-epileptic agents such as anti-convulsants, psychotropic agents, ion channel blockers, etc. may be injected into or around the site of abnormal electrical activity to provide for the time-release of the active agent at the particular site. This treatment eliminates or minimizes the side of effects of more systemic form of treatment such as oral administration of an antiepileptic medication.

In another aspect, cardiac arrhythmias may be treated using the inventive system. A patient suffering from or at a risk of cardiac arrhythmias may be treated using the inventive composition by injecting an anti-arrhythmic agent at the site of the abnormal electrical activity or along the conduction pathway of such unwanted electrical activity. The microparticles of the inventive system used in treating cardiac arrhythmias may be loaded with any agent known to decrease, inhibit, or eliminate unwanted electrical activity such as ion channel blocks, receptor antagonists, etc. The microparticles once delivered are designed to release the active agent over days to weeks to months. For prolonged prevention of cardiac arrhythmias repeated injection may be necessary. The delivery of the microparticles to the affected site may be aided by the use of imaging studies, electrical conduction studies, electrical activity, ECG, etc. The time release of the active agent at the affected site within the heart or its conduction system may lead to more effective treatment and prevention of cardiac arrhythmias without many of the side effects of other current treatments which include invasive surgery, oral medication, and pacemaker implants.

In yet another aspect, the inventive system is employed to prevent or treat pre-term labor. Tocolytic agents may be encapsulated in the microparticles that are then administered into the uterus or near the uterus of the expecting mother. The time release of the tocolytic agent inhibits or decreases the electrical activity leading to unwanted uterine contractions. In treating pre-term labor, the risk to the fetus is minimized. The inventive system provides a more effective and localized treatment of pre-term labor.

### DEFINITIONS

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a transgenic animal.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Biocompatible”: The term “biocompatible”, as used herein is intended to describe compounds that are not toxic to cells. Compounds are “biocompatible” if their addition to cells in vitro results in less than or equal to 20% cell death and do not induce inflammation or other such adverse effects in vivo.

“Biodegradable”: As used herein, “biodegradable” compounds are those that, when introduced into cells, are broken down by the cellular machinery into components that the cells can either reuse or dispose of without significant toxic effect on the cells (i.e., fewer than about 20% of the cells are killed).

“Effective amount”: In general, the “effective amount” of an active agent or the microparticles refers to the amount necessary to elicit the desired biological response. As will be appreciated by those of ordinary skill in this art, the effective amount of microparticles may vary depending on such factors as the desired biological endpoint, the agent to be delivered, the composition of the encapsulating matrix, the target tissue, etc. For example, the effective amount of microparticles containing an anti-epileptic agent to be delivered is the amount that results in a reduction in the severity or frequency of seizures and/or unwanted electrical activity. In another example, the effective amount of microparticles containing an anti-arrhythmic medication to be delivered to the heart of the individual is the amount that results in a decrease in the amount or frequency of the unwanted electrical activity, or decrease in clinical signs (e.g., ECG findings) or symptoms (e.g., syncopal episodes) of cardiac arrhythmias.

“Peptide” or “protein”: According to the present invention, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Known naturally-occurring small molecules include, but are not limited to, penicillin, erythromycin, taxol, cyclosporin, and rapamycin. Known synthetic small molecules include, but are not limited to, ampicillin, methicillin, sulfamethoxazole, and sulfonamides.

“Sugar”: The term “sugar” refers to any carbohydrate. Sugars useful in the present invention may be simple or complex sugars. Sugars may be monosaccharides (e.g., dextrose, fructose, inositol), disaccharides (e.g., sucrose, saccharose, maltose, lactose), or polysaccharides (e.g., cellulose, glycogen, starch). Sugars may be obtained from natural sources or may be prepared synthetically in the laboratory. In a preferred embodiment, sugars are aldehyde- or ketone-containing organic compounds with multiple hydroyxl groups.

“Surfactant”: Surfactant refers to any agent which preferentially absorbs to an interface between two immiscible phases, such as the interface between water and an organic solvent, a water/air interface, or an organic solvent/air interface. Surfactants usually possess a hydrophilic moiety and a hydrophobic moiety, such that, upon absorbing to microparticles, they tend to present moieties to the external environment that do not attract similarly-coated particles, thus reducing particle agglomeration. Surfactants may also promote absorption of a therapeutic or diagnostic agent and increase bioavailability of the agent. The term surfactant may be used interchangeably with the terms lipid and emulsifier in the present application.

### BRIEF DESCRIPTION OF THE DRAWING

FIG. 1 is a scanning electron micrograph of spray-dried lipid-protein particles loaded with 0.2% (w/w) muscimol.

FIG. 2 shows the in vitro release of muscimol from the 0.2% (w/w) muscimol particles. Data points are medians with standard deviations.

FIG. 3 shows the mean seizure scores with standard deviations of rats given pilocarpine following administration of various treatments (n=4 in all groups).

FIG. 4 shows representative examples of cell loss in the hippocampus (A, B-CA3 subfield; C,D-CA1 subfield of rats given pilocarpine after 5 μg of encapsulated (A, C) or unencapsulated (B, D) muscimol. Images A and C were from a rat that did not have seizures whiles images B and D were from a rat that did. Note cell loss as indicated by arrows. Brains were stained with thionin. Calibration =50 μm.

FIG. 5 is a representative example of Timm staining in hippocampus sections (A, B-CA3 subfield; C, D, E-hilus). A and C are from rats given pilocarpine after 5 μg of encapsulated muscimol. The rat in B and D received unencapsulated muscimol while the rate in E received normal saline prior to pilocarpine. Images A and C were from a rat that did not have a seizure while images B, D, and E were from rats that did. Note sprouting (arrow) in the CA3 subfield in B. Sprouting of mossy fibers was also seen in the inner molecular layer (IML) of the dentate granule cells (DGC) in the two rats with seizures (arrows in D and E). Brains were stained with Timm stain. Calibration=100 μm.

### DETAILED DESCRIPTION OF CERTAIN PREFERRED EMBODIMENTS OF THE INVENTION

The present invention provides a controlled release system for administering pharmaceutical agents to an electrically excitable tissue in order to prevent or decrease undesired electrical activity. Decreasing, eliminating, or decreasing the risk of the aberrant electrical activity creates benefits to the patient in terms of curing, diminishing, or preventing a disease state. In particular, the inventive system may be used to deliver drugs useful in treating epilepsy to the brain, cardiac arrhythmias to the heart or its conduction system, and pre-term labor to the uterus.

Controlled Release System

Any controlled release system for delivering a pharmaceutical agent may be used in the inventive systems and methods. As would be appreciated by one of skill in the art, the controlled release system used in treating a particular disease in a particular patient will depend on many factors. These factors may include the rate of release of the agent being delivered, the agent being delivered, the composition of the controlled release system, the disease state being treated, the site of administration, the status of the patient, the need for repeat administration, etc. A health care professional such as a physician would be able to weigh these factors and tailor the treatment regimen to the individual suffering from a particular disease.

In one embodiment, microparticles are used to deliver the pharmaceutical agent in a controlled release formulation. Any type of microparticle known in the art can be used to deliver the desired pharmaceutical agent to the diseased tissue or site with the unwanted electrical activity. The type of microparticles useful in the inventive system include liposomes, spray-dried particles, microspheres formed by single- and double-emulsion techniques, microparticles produced off of a micropatterned surface, and microparticles formed by spray drying, coacervation, or spontaneous emulsification. The microparticles are typically less than 1 mm in size. Preferably the microparticles are less than 0.5 mm in size; and more preferably, less than 0.1 mm in size. In certain preferred embodiments, the microparticles are between about 1 micron and about 100 microns in size. Preferably, the microparticles are small enough so that they can be suspended in a suitable carrier (e.g., water, saline solution, dextrose solutions, carboxymethylcellulose, mannitol, buffered solutions, etc.) and injected through a needle into the site of administration.

The microparticles may be composed of any polymer or substance used in the preparation of microparticles. Preferably, the microparticles are biocompatible and/or biodegradable. In certain embodiments, the microparticles are made of polyesters such as poly(glycolide-co-lactide) (PLGA), polyglycolic acid, poly-β-hydroxybutyrate, and polyacrylic acid ester. In other embodiments, the microparticles are lipid-protein-sugar particles (LPSPs). For a more detailed description of the LPSP particles and their preparation and uses, see U.S. patent applications 60/240,698; 60/240,636; Ser. No. 09/981,020; Ser. No. 09/981,460; each of which is incorporated herein by reference. In certain embodiments, LPSPs include those made from dipalmitoylphosphatidyl-choline, albumin, and lactose. Preferred ratios of the three components include 50-70% of the lipid, 10-30% sugar, and 10-30% protein. In addition, a fraction (e.g., 0.1%-5%) of the particle may be composed of a high-molecular weight, hydrophobic, amphiphilic, or charged molecule to alter (e.g., generally prolong) the release kinetics of the particle. The active agent may be present in the microparticle between 0.001% and 10%, preferably between 0.1% and 5%, and more preferably between 1% and 3%. In certain embodiments, for example, when an immunogenic epitope or growth factor is the active agent, minute quantities on the order of 0.001% may be included in the microparticle. In other embodiments, the active agent may be present in the particle in excess of 90%. As would be appreciated by one of skill in the art, the composition of the microparticles will depend on the time-release schedule desired in the particular application. In certain embodiments, a mixture of microparticles with different half-lives and other controlled release properties will be used to achieve the release profile needed in a specific application. For example, a mixture may contain particles with a half life of 24 hrs., 48 hrs., 72 hrs., and 96 hrs. to get a steady release of active agent over the course of a week. To give but one example of a particular application, for long term seizure control, a composition of particles with a long half-life will be necessary to avoid multiple repeated administrations.

In addition to microparticles, other controlled release systems may be used including gels, gel/sols including viscous gels (e.g., carboxymethylcellulose), liquids with reverse thermal gelation properties (e.g., substances that are liquid at room temperature and gel at body temperature such as poloxamer 407). These other controlled release systems may be used separately or in conjunction with particles.

Agents

The pharmaceutical agents delivered using the controlled release systems as described above include any agent useful in treating the disease of the patient. For example in the case of treating epilepsy, any pharmaceutical agent used to treat or prevent epilepsy may be used in the inventive system. In the case of cardiac arrhythmias, any agent useful in the treatment or prevention of cardiac arrhythmias may be used in the inventive system. Also, in the case of treating pre-term labor, any agent useful in stopping or preventing pre-term labor may be used in the inventive system. Preferably administering the agent at the site of disease leads to less side effects to the patient than if one were to deliver the agent systemically. In certain cases, the dose needed to deliver the agent systemically and yield a therapeutic effect could not be used because the agent is too toxic or would cause side effects that are unacceptable such as a high risk of death.

An illustrative list of pharmaceutical agents useful in the treatment of epilepsy includes site 1 sodium channel blockers such as tetrodotoxin (TTX), saxitoxin (STX), neosaxitoxin, decarbamoyl saxitoxin, gonyautoxin, and derivative thereof; conventional local anesthetics such as benzocaine, bupivacaine, cocaine, etidocaine, lidocaine, mepivacaine, pramoxine, prilocaine, procaine, proparacaine, ropicaine, tetracaine, procainamide, and dibucaine; other compounds with local anesthetic properties such as vanilloid receptor agonists such as capsaicin or resiniferatoxin; natural or synthetic glucocorticoid receptor agonists such as hydrocortisone, dexamethasone, cortisone, prednisone, beclomethasone, betamethasone, flunisolide, methyl prednisone, paramethasone, prednisolone, triamcinolome, alclometasone, amcinonide, clobetasel, fludrocortisone, diflurosone diacetate, flucinolone acetonide, fluoromethalone, flurandrenolide, halcinonide, medrysone, and mometasone; and specific anti-epileptic drugs such as phenytoin, benzodiazepines, valproic acid, carbazepine, felbamate, and barbiturates. In certain embodiments, more than one of the above listed agents are included a particular microparticle or a mixture of microparticles containing different agents is administered. An illustrative, but not exhaustive, list of antiseizure agents include carbamaepine, phenytoin, phenobarbital, primidone, valproate, gabapentin, lamotrigine, clonazepam, and ethosuximide. In certain embodiments, the inventive controlled release system utilizes a site 1 sodium channel blocker, a local anesthetics, and dexamethasone, singly or in combination with each other or other agents listed above.

With respect to the treatment of cardiac arrhythmias, the agents may include any agent useful in the treatment or prevention of cardiac arrhythmias including phenytoin, adrenergic antagonists, amiodarone, procaiamide, lidocaine, bretylium, adenosine, verapamil, beta-blockers such as propranolol and sotalol, magnesium salts such as magnesium sulfate, isoproterenol, tocainide, quinidine, disopyramide, moricizine, propafenone, flecainide, diltiazem, digitalis, digoxin, digitoxin, esmolol, mexiletine, moricizine, phenytoin, propafenone., etc. In certain embodiments, the inventive controlled release system for treating cardiac arrhythmias utilizes a site 1 sodium channel blocker, a local anesthetics, and dexamethasone, singly or in combination with each other or other agents listed above.

Tocolytic agents useful in the treatment of magnesium sulfate, β₂-adrenergic receptor agonists, calcium channel blockers, oxytocin antagonists, and prostaglandin synthetase inhbitors. To give but a few illustrative examples of tocolytic agents, the agents used in the controlled release systems of the present invention include ritodrine hydrochloride, terbutaline, fenoterol, albuterol, magnesium sulfate, nifedpine, and indomethacin. In certain embodiments, the inventive controlled release system for use in the treatment of pre-term labor utilizes a site 1 sodium channel blocker, a local anesthetics, and dexamethasone, singly or in combination with each other or other agents listed above. In other embodiments, the controlled release system includes a site 1 sodium channel blocker with or without a local anesthetic. In yet other embodiments, the controlled release system includes a site 1 sodium channel blocker with or without dexamethasone. In certain embodiments, the use of local anesthetics alone or local anesthetics plus dexamethasone as the active agent(s) in a controlled release formulation are not included in the present invention.

Targeting Agents

The microparticles used in the inventive system may be modified to include targeting agents since it is often desirable to target a microparticle to a particular cell, collection of cells, tissue, or organ. A variety of targeting agents that direct pharmaceutical compositions to particular cells are known in the art (see, for example, Cotten et al. Methods Enzym. 217:618, 1993; incorporated herein by reference). The targeting agents may be included throughout the particle or may be only on the surface. The targeting agent may be a protein, peptide, carbohydrate, glycoprotein, lipid, small molecule, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gp120 envelope protein of the human immunodeficiency virus (HIV), carbohydrates, receptor ligands, sialic acid, etc. If the targeting agent is included throughout the particle, the targeting agent may be included in the mixture that is spray dried to form the particles. If the targeting agent is only on the surface, the targeting agent may be associated with (i.e., by covalent, hydrophobic, hydrogen boding, van der Waals, or other interactions) the formed particles using standard chemical techniques.

Pharmaceutical Compositions

Once the microparticles have been prepared, they may be combined with other pharmaceutical excipients to form a pharmaceutical composition. As would be appreciated by one of skill in this art, the excipients may be chosen based on the route of administration as described below, the agent being delivered, time course of delivery of the agent, etc.

Pharmaceutical compositions of the present invention and for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In a particularly preferred embodiment, the LPSPs are suspended in a carrier fluid comprising 1% (w/v) sodium carboxymethyl cellulose and 0.1% (v/v) Tween 80.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Methods of Making Microparticles

The microparticles useful in the inventive system may be prepared using any method known in this art. These include spray drying, single and double emulsion solvent evaporation, solvent extraction, phase separation, simple and complex coacervation, and other methods well known to those of ordinary skill in the art. A particularly preferred method of preparing the particles is spray drying. The conditions used in preparing the microparticles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the matrix.

Methods developed for making microparticles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283, 1987; Mathiowitz et al. J. Appl. Polymer Sci. 35:755-774, 1988; each of which is incorporated herein by reference).

If the particles prepared by any of the above methods have a size range outside of the desired range, the particles can be sized, for example, using a sieve.

As mentioned above, LPSPs are preferably prepared by spray drying. Prior methods of spray drying, such as those disclosed in PCT WO 96/09814 by Sutton and Johnson (incorporated herein by reference), provide the preparation of smooth, spherical microparticles of a water-soluble material with at least 90% of the particles possessing a mean size between 1 and 10 micrometers. The method disclosed by Edwards et al. in U.S. Pat. No. 5,985,309 (incorporated herein by reference) provides rough (non-smooth), non-spherical microparticles that include a water-soluble material combined with a water-insoluble material. Any of the methods described above may be used in preparing the inventive LPSPs. Specific methods of preparing LPSPs containing muscimol are described below in the Examples.

Application

The controlled release delivery systems useful in the present invention loaded with the appropriate pharmaceutical agent or agents can be used to control the deleterious effect of aberrant or normal (physiological) electrical activity in excitable tissues (for formulations useful in providing local anesthesia, see U.S. Pat. No. 6,326,020; incorporated herein by reference). The release of the agent from a controlled release delivery system may decrease, prevent, or eliminate the undesired electrical activity in the excitable tissue or organ; however, the agent may also just ameliorate signs, symptoms, and effects of the aberrant electrical activity without directly affecting the electrical activity. Therefore, in certain embodiments, the agent may achieve a physiological goal (e.g., normal cardiac rhythm, tocolysis, or seizure control) even in the presence of undesired or desired electrical activity in the excitable tissue.

The body has many excitable tissue which are amenable to the inventive system. These exemplary list of organs and tissues include nerves, skeletal muscle, smooth muscle, cardiac muscle, uterus, central nervous system, spinal cord, brain, retina, olfactory system, auditory system, skin, etc. In certain embodiments, the inventive system and methods are used to control aberrant electrical activity in the brain, spinal cord, or central nervous system. For example, epilepsy may be treated using the inventive system. In another embodiment, cardiac arrhythmias may be controlled or prevented using the inventive system. And in yet another embodiment, tocolysis in a patient suffering from pre-term labor may be achieved using the inventive system.

As would be appreciated by one of skill in this art, the first step in treating a patient is identifying a patient suffering from a disease state which includes or is characterized by aberrant electrical activity. For example, this may include diagnosing a person with epilepsy by EEG studies, by clinical signs and symptoms, history, etc. Diagnosing a patient with cardiac arrhythmias may include ECG studies, history, physical exam, Holter monitoring, etc. Diagnosing a pregnant women in preterm labor may include physical exam, history, ultrasound, etc. Once a proper diagnosis of the disease has been made, one of skill in this art probably a licensed physician can determine the treatment to be administered. The treatment may include the inventive method as described herein. Additionally, the treatment may include the inventive method and another treatment regimen such as oral medication.

For certain disease condition such as epilepsy and cardiac arrhythmias where a portion of the excitable organ is involved, it may be necessary to localize the affected area so that the inventive compositions with the therapeutic agent can be delivered into or very close to the diseased area. In this way, the pharmaceutical agent does not affect non-diseased areas of the excitable organ and may also help to avoid unwanted side effects. To give but an illustrative example, in the case of a patient suffering from seizures, it may be useful to determine where in the brain the seizure are originating. This may be done by EEG. Then the inventive composition is delivered to the affected area to prevent seizure activity. Localizing the origin of aberrant electrical activity in the heart and directing therapy to that site may also be useful in the treatment of a patient suffering from cardiac arrhythmias. The site in the heart or its conduction system may be identified by EKG or other electrical studies of the heart.

In certain embodiments, the inventive composition is delivered near the affected site, or in another embodiment, the administered inventive composition provides a systemic depot for the pharmaceutical agent. Therefore, depending on the disease and the patient, the physician may decide to administer the inventive composition into, nearby, or away from the affected site. The site of administration may include a site nearby the target organ rather than actually in the organ.

Once the site of administration has been determined, the type (e.g., microparticles, microspheres, sol/gel, gels, etc.) and composition (e.g., excipient, polymers, agent) of the controlled delivery system must be determined. The attending physician treating the patient may weight such factors as the status of the patient, the disease be treated, the time course of the treatment, the agent to be delivered, etc. to determine the composition to be delivered. For example, in the case of a patient suffering from epilepsy who needs long term medication. A microparticle composition with a long half life may be chosen so as to minimize repeated administration of the composition. For example, the microparticles may have a half-life of months and may also release a pharmaceutical agent with a long half-life. In contrast, in treating a pregnant woman in pre-term labor who is near the end of her pregnancy, it may be useful to only provide tocolytic therapy for a few days to a couple of weeks. In this case, microparticles with a half-life of 24 hrs. to 48 hrs. may be used.

In certain patients, repeat administration of the inventive composition will be necessary to further treat or prevent the diseased state in the future. In the case of cardiac arrhythmias or epilepsy, the treatment may need to be life-long. The inventive treatment method may also be combined with more conventional treatment modalities such as oral medication, pacemaker implantation, etc.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Effectiveness of Muscimol-containing Microparticles Against Pilocarpine-induced Focal Seizures

Introduction

Oral pharmacotherapy is the cornerstone of the treatment of chronic seizure disorders. Antiepileptic drugs are typically administered multiple times daily; the dosage and frequency of administration are determined by the pharmacokinetic characteristics of the drugs and their systemic side effects (Cloyd, J. C., Remmel, R. P. Antiepileptic drug pharmacokinetics and interactions: impact on treatment of epilepsy. Pharmacotherapy 2000; 20: 139S-151S; French J A, Gidal B E. Antiepileptic drug interactions. Epilepsia 2000; 41: S30-S36; each of which is incorporated herein by reference). The dose of systemically delivered drug required to achieve a brain concentration sufficient to control seizures may result in unacceptable side effects (Perucca E, Dulac O, Shorvon S, Tomson T. Harnessing the clinical potential of antiepileptic drug therapy: dosage optimization. CNS Drugs 2001; 15: 609-621; Swann A C. Major system toxicities and side effects of anticonvulsants. J. Clin. Psychiatry 2001; 62: 16-21; each of which is incorporated herein by reference). This is particularly true in some forms of epilepsy (e.g., epilepsia partialis continua), in which seizure activity can be unrelenting. The sequelae of the disorder and the treatment (barbiturate coma, neurosurgery) can be severe. A drug delivery system that could directly target the epileptic region in the brain would offer enormous advantages, especially since approximately 60% of seizures are partial in nature (Hauser W A, Kurland L T. The epidemiology of epilepsy in Rochester, Minn., 1935 through 1967. Epilepsia 1975; 16: 1-66; Hauser W A, Hersdorffer D C. Epilepsy: Frequency, causes and consequences. New York: Demos, 1990; each of which is incorporated herein by reference). Furthermore, status epilepticus is most likely to occur in patients with partial seizures (Hauser W A. Status epilepticus: epidemiologic considerations. Neurology 1990; 40: 9-13; incorporated herein by reference).

The effectiveness of focally delivered anticonvulsants in treating experimental models has been demonstrated (Eder H G, Jones D B, Fisher R S. Local perfilsion of diazepam attenuates injterictal and ictal events in the bicuculline model of epilepsy in rats. Epilepsia 1997; 38: 516-521; incorporated herein by reference). Automated systems using a catheter at the epileptogenic focus have been devised that are effective in terminating induced seizures (Stein A G, Eder H G, Blum D E, Drachev A, Fisher R S. An automated drug delivery system for focal epilepsy. Epilepsy Res. 2000; 39: 103-114; incorporated herein by reference). Relatively large implants impregnated with various agents (Graber K D, Prince D A. Tetrodotoxin prevents posttraumatic epileptogenesis in rats. Ann. Neurol. 1999; 46: 234-242; Kubek A J, Liang D, Byrd K E, Domb A J. Prolonged seizure suppression by a single implantable polymeric-TRH microdisk preparation. Brain Res. 1998; 809: 189-197; Tamargo R J, Rossell L A, Kossoff E H, Tyler B M, Ewend M G, Aryanpur J J. The intracerebral administration of phenytoin using controlled-release polymers reduces experimental seizures in rats. Epilepsy Res 2002; 48: 145-155; each of which is incorporated herein by reference) are also effective in animal studies.

In this Example, the effect of intrahippocampal injection of biodegradable and biocompatible lipid-protein-sugar particles loaded with an anticonvulsant drug in the prevention of seizures in a rat model is examined. These microparticles are generally several microns in diameter, suspend readily in physiological carrier fluids (Kohane D S, Lipp M, Kinney R, Lotan N, Langer R. Sciatic nerve blockade with lipid-protein-sugar particles containing bupivacaine. Pharm. Res. 2000; 17: 1243-1249; incorporated herein by reference), and can be injected stereotactically through a small gauge catheter or needle (Kohane D S, Plesnila N, Thomas S S, Le D, Langer R S, Moskowitz M A. Lipid-sugar particles for intracranial drug delivery: safety and biocompatibility. Brain Res. 2002; (in press); incorporated herein by reference). Such particles have been used for drug delivery to the peripheral (Kohane D S, Lipp M, Kinney R, Lotan N, Langer R. Sciatic nerve blockade with lipid-protein-sugar particles containing bupivacaine. Pharm. Res. 2000; 17: 1243-1249; incorporated herein by reference) and central (Kohane DS, Plesnila N, Thomas S S, Le D, Langer R S, Moskowitz M A. Lipid-sugar particles for intracranial drug delivery: safety and biocompatibility. Brain Res. 2002; (in press); incorporated herein by reference) nervous systems. They can also be engineered to contain a wide variety of drugs and excipients, and to provide varying rates of drug release. They are biocompatible in the epineurium in the rat peripheral nervous system (Kohane D S, Lipp M, Kinney R, Anthony D, Lotan N, Langer R. Biocompatibility of lipid-protein-sugar particles containing bupivacaine in the epineurium. J. Biomed. Mat. Res. 2002; 59: 450-459; incorporated herein by reference), and the murine brain (Kohane D S, Plesnila N, Thomas S S, Le D, Langer R S, Moskowitz M A. Lipid-sugar particles for intracranial drug delivery: safety and biocompatibility. Brain Res. 2002; (in press); incorporated herein by reference).

Our model of epilepsy is hippocampal injection of pilocarpine (Turski L, Ikonomidou C, Turski W A, Bortolotto Z A, Cavalheiro E A. Review: cholinergic mechanisms and epileptogenesis. The seizures induced by pilocarpine: a novel experimental model of intractable epilepsy. Synapse 1989; 3: 154-171; Millan M, Chapman A G, Meldrum B S. Extracellular amino acid levels in hippocampus during pilocarpine-induced seizures. Epilepsy Res 1993; 14: 139-148; each of which is incorporated herein by reference), a nonselective muscarinic agonist. This model has been shown to bear histological similarities to temporal lobe epilepsy in humans (Isokawa M, Mello L E. NMDA receptor-mediated excitability in dendritically deformed dentate granule cells in pilocarpine-treated rats. Neurosci. Lett. 1991; 129: 69-73; incorporated herein by reference). Muscimol, a potent GABA_{A} receptor agonist anticonvulsant (Collins R C. Anticonvulsant effects of muscimol. Neurology 1980; 30: 575-581; incorporated herein by reference), was the anticonvulsant used.

Materials and methods

Animal Care

Sprague Dawley rats (Charles River Laboratories, Cambridge, Mass.) (n=21), weighing 200-250 gm at surgery were maintained in a 12 hr light/dark cycle. Animals had access to food and water ad libitum. All procedures were approved by the Animal Care Committee of Children's Hospital and were in accordance with guidelines set by the National Institutes of Health.

Preparation of Lipid-protein-sugar Particles (LPSPs)

Dipalmitoylphosphatidyl-choline (DPPC; Avanti Polar Lipids, Alabaster, Ala.) was dissolved in ethanol; albumin, lactose, and muscimol (all from Sigma Chemical Co., St. Louis, Mo.) were dissolved in water. The two solution were mixed (so the final proportion (w/w) of solutes was DPPC 59.3: albumin 19.3: lactose 19.3: muscimol 2), and spray-dried using a Model 190 bench top spray drier (Büchi Co, Switzerland) as described (Kohane D S, Lipp M, Kinney R, Lotan N, Langer R. Sciatic nerve blockade with lipid-protein-sugar particles containing bupivacaine. Pharm. Res. 2000; 17: 1243-1249; incorporated herein by reference). Blank particles were produced in an identical manner, except that muscimol was not included, and the amounts of the inactive excipients were increased accordingly.

Particle Size and Shape Determination.

Particle size was determined with a Coulter Multisizer (Coulter Electronics Ltd., Luton, U.K.), using a 30 μm orifice. Surface characteristics of particles were determined by scanning electron microscopy on an AMR-1000 (Amray Inc., Bedford, Mass.). Samples were mounted on stubs and given a gold-palladium conductive coating, and scanned at 10 kV.

Muscimol Content of Particles.

The actual muscimol content of particles was determined by dissolving a known quantity of particles in 1 ml methanol, adding HPLC running buffer (see below) to a total volume of 5 ml, centrifugating the solution at 14,000 rpm for 10 minutes, and measuring the muscimol concentration in the supernatant, using a standard curve.

Muscimol Release.

Twenty-five mg of particles containing 500 mcg of muscimol were suspended in phosphate buffered saline (PBS) pH 7.4 and placed in a dialysis tube with an 8,000 MW cut-off (Spectra/Por 1.1 Biotech Dispo-dialyzer). The tube was then submerged in 12 ml of PBS and incubated at 37 degrees C. At predetermined intervals, the external PBS was removed for analysis by HPLC (see below), and replaced by fresh PBS.

HPLC.

HPLC assays were performed on a HP 1100 HPLC system. Samples in 50 μl volume were injected onto a 4.6 mm (ID)×25 cm (L) Spherisorb ODS-2 column (Column Engineering, Ontario, Calif.). The column was eluted with an aqueous solution of 0.5% v/v HBTA (Heptafluorobutyric acid, Fluka) at 1 ml/min. Muscimol was detected by an UV detector with absorbance wavelength set at 230 nM.

Induction of Seizures

The method is similar to that described by Cavalheiro et al. (Cavalheiro E A, Czuzwar S J, Kleinrok S. Intracerebral cholinomimetics produce seizure-related brain damage in rats. Brit. J. Pharmacol. 1983; 79: 284P; incorporated herein by reference). Animals were anesthetized with pentobarbital (40 mg/kg) and an electrode-cannula guide (Plastic one, USA) was stereotactically implanted in the CA3 region of the dorsal hippocampus (lateral from midline 3.5 mm, posterior from bregma 3.8 mm, and 3.8 mm deep from skull; FIG. 35 in (Paxinos G, Watson C. The rat brain in stereotaxic coordinates, 4th edition. San Diego: Academic Press, 1998; incorporated herein by reference)). The electrode-cannula guide was fixed to skull using dental cement and skull screws. After surgery, a dummy cannula was placed into the guide cannula to prevent occlusion. One week later the rats received 30 μl of one anticonvulsant or control treatment (encapsulated or free) through the cannula using a microinfusion pump (Baxter, model AS40A) for 15 min at a rate of 0.12 ml/hr. Thirty minutes after completion of these treatments, 40 mM pilocarpine was infused for 50 min at a rate of 0.12 ml/hr. Subsequently, continuous EEGs were recorded from the electrode-cannulae, and the animals were videotaped for a minimum of two hours. Animals were then kept in the laboratory for six hours under close visual observation and were returned to the vivarium only after the animal was seizure-free for an hour. Behavior was coded every 10 minutes using the scale in Table 1. All procedures and observations were done by a single observer between 6 AM and 6 PM. Four animals were used in each treatment group, except as specified in the text.

TABLE 1
Seizure scoring system

| Score | Behavior |
|---|---|
| 0 | Normal behavior |
| 1 | Motionless, staring |
| 2 | Chewing |
| 3 | Forelimb clonus |
| 4 | Bilateral forelimb clonus |
| 5 | Bilateral forelimb clonus with rearing |
| 6 | Tonic posturing |

Histology

Two weeks after induction of seizure, animals were sacrificed and prepared for routine histology and Timm histochemistry. Following deep anesthesia with sodium pentobarbital (100 mg/kg), rats were perfused transcardially with 300 ml sodium sulfide medium (2.295 g Na₂S, 2.975 g NaH₂PO₄/H₂O in 500 ml H₂O) followed by 300 ml 4% paraformaldehyd were postfixed in 4% paraformaldehyde for 24 h and then placed in a 30% sucrose solution until they sank to the bottom of the vial. Coronal sections through the entire extent of the hippocampus were cut at 40 μm on a freezing microtome and sections were stored in phosphate buffered saline. Every fourth section was stained for mossy fibers using Timm stain as follows. The sections were developed in the dark for 40-45 min in a solution of 50% Arabic gum (120 ml), 10 ml of citric acid (51 g/100 ml H₂O), 10 ml sodium citrate (47 g/100 ml H₂O), 3.47 g hydroquinone in 60ml, and 212.25 mg AgNO₃. Following washing, the slides were dehydrated in alcohol, cleared in xylene, and mounted on slides with Permount (Fisher Scientific, Pittsburgh, Pa.). Timm staining was analyzed using a scoring system (0 to 5) for terminal sprouting in the CA3 and supragranular regions (Holmes G L, Gaiarsa J L, Chevassus-Au-Louis N, Ben-Ari Y. Consequences of neonatal seizures in the rat: morphological and behavioral effect. Ann. Neurol. 1998; 44: 845-857; Holmes G L, Sarkisian M, Ben-Ari Y, Chevassus-Au-Louis N. Mossy fiber sprouting following recurrent seizures during early development in rats. J. Comp. Neurol. 1999; 404: 537-553; each of which is incorporated herein by reference). In addition, another series of sections were stained with thionin to assess cell number and architecture (Mikati M A, Holmes G L, Chronopoulos A, et al. Phenobarbital modifies seizure-related brain injury in the developing brain. Ann. Neurol. 1994; 36: 425-433; incorporated herein by reference). Slides were analyzed for cell loss in the CA3, CA1 and hilar region using a semi-quantitative visual scoring system (0 to 5) (Mikati M A, Holmes G L, Chronopoulos A, et al. Phenobarbital modifies seizure-related brain injury in the developing brain. Ann. Neurol. 1994; 36: 425-433; incorporated herein by reference). Cell loss was assessed both ipsilateral and contralateral to the injection site. Five brain slices per rat (ten hippocampal sections in total) were examined and these scores were added to obtain a total score for each region. This score was divided by ten to come to a mean score per hippocampus per rat. All scoring was done by an investigator blinded to treatment group (GLH).

Statistical Analysis

The Kolmogorov-Smirnov goodness-of-fit test was used to assess normality (Gaussian-shaped distribution) for all continuous variables. Two-way repeated-measures analysis of variance (ANOVA) was used to compare behavior scores between normal saline, free muscimol, and encapsulated muscimol groups with the F-test for interaction used to assess differences in trajectories over the 120 minute time course following pilocarpine injection (Sokal R R, Rohlf F J. Biometry: The Principles and Practice of Statistics in Biological Research. 3d Ed. edition. New York: W. H. Freeman, 1995, pp 498-524; incorporated herein by reference). A Bonferroni adjusted value of p<0.017 (0.05/3) was considered statistically significant to account for multiple group comparisons. Factorial analysis of variance (ANOVA) with post-hoc multiple comparisons by Fisher's least significant difference (LSD) procedure was used to evaluate total cell loss and Timm scores (CA3 and supragranular regions) between muscimol, LPSP, and normal saline treatment groups (Montgomery DC. ‘In:’Design and analysis of experiments. 5th edition. New York: John Wiley & Sons, 2001, pp 96-104; incorporated herein by reference). Cell loss and sprouting of Timm fibers were compared between pilocarpine-induced status epilepticus and non-seizure rats by unpaired Student t-tests. Data are presented in terms of the mean and standard deviation (SD). Statistical analysis was performed using the SAS software package (Version 6.12, SAS Institute, Cary, N.C.). All reported p-values are two-tailed.

Results

Characteristics of Muscimol-containing and Blank Particles

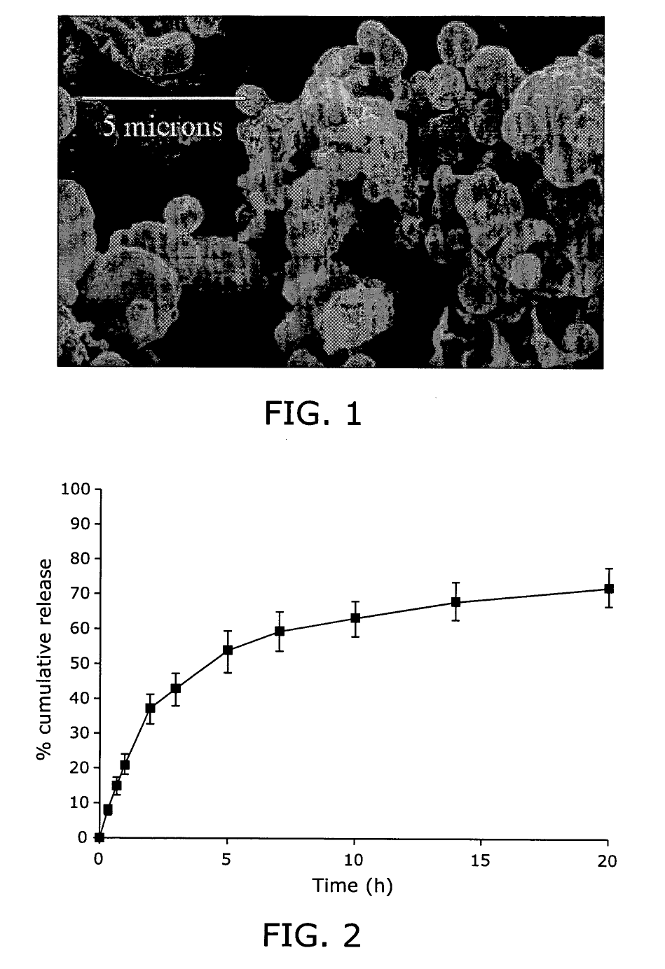

Lipid-protein-sugar particles (LPSPs) were produced as a fine white dry powder. By electron microscopy, blank and muscimol-loaded LPSPs were generally spherical in shape (FIG. 1). The median volume-weighted diameter was 4 to 5 μm (i.e., even though the majority of particles were smaller—approximately 1 μm—the larger particles contributed proportionally more to the total volume of material). Typical yields from each production run were 30 to 40% of total solute. Actual loading of particles was verified by HPLC, and was found to be equal to the theoretical loading (20 μg of muscimol per mg of particle, or 2% (w/w)). Muscimol release from samples of particles was measured as per Methods (FIG. 2), with complete release occurring over 5 days.

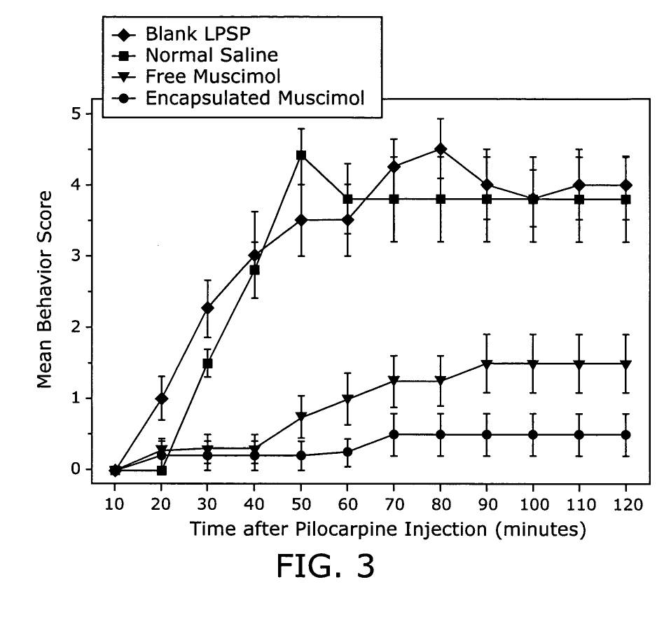

Effect of Particles Against Focal Pilocarpine-induced Seizures (FIG. 3).

In animals in which pilocarpine injection was preceded by administration of normal saline (i.e., no muscimol), seizure activity was stereotyped. Around 10 to 15 minutes after pilocarpine injection, rats became immobile with minimal facial movements and staring. This was followed by chewing salivation which then progressed into forelimb clonus, occurring either unilaterally or bilaterally. Eventually the animal had bilateral forelimb clonus with rearing. The final stage of the seizure was tonic posturing. Following the tonic phase the animals returned to early seizure stages. For example, tonic activity would be interspersed with forelimb clonus, chewing, and immobility.

Repeated-measures ANOVA indicated a significant overall difference in mean behavior scores between animals receiving normal saline, 5 μg of unencapsulated (free) muscimol, or 5 μg of encapsulated muscimol prior to pilocarpine, F(2,9)=11.95, p<0.001. Multiple comparisons revealed that the rise of the trajectory in behavior score over the 120 minute time course following pilocarpine injection was significantly faster in the normal saline group compared to the free muscimol (F=4.52, p<0.001) and encapsulated muscimol (F=7.39, p<0.001) groups, indicating that muscimol reduces seizure activity as measured by behavior score. The increase in behavior score was significantly faster (steeper slope) for animals in the free muscimol compared to the encapsulated muscimol group (F=2.68, p<0.01), suggesting that encapsulation enhances the protective antiepileptic effect of muscimol. Blank particles did not exert an anti-epileptic effect: there was no significant difference in the change in seizure scores over time between animals receiving blank LPSPs and normal saline over the 120 minutes after pilocarpine injection (F=0.44, p=0.69).

Animals receiving particles containing 10 μg (n=4) and 20 μg (n=1) prior to administration of pilocarpine did not experience seizures. We did not pursue additional experiments in these groups because of the high mortality in the unencapsulated comparison groups, presumably from the side effects of muscimol.

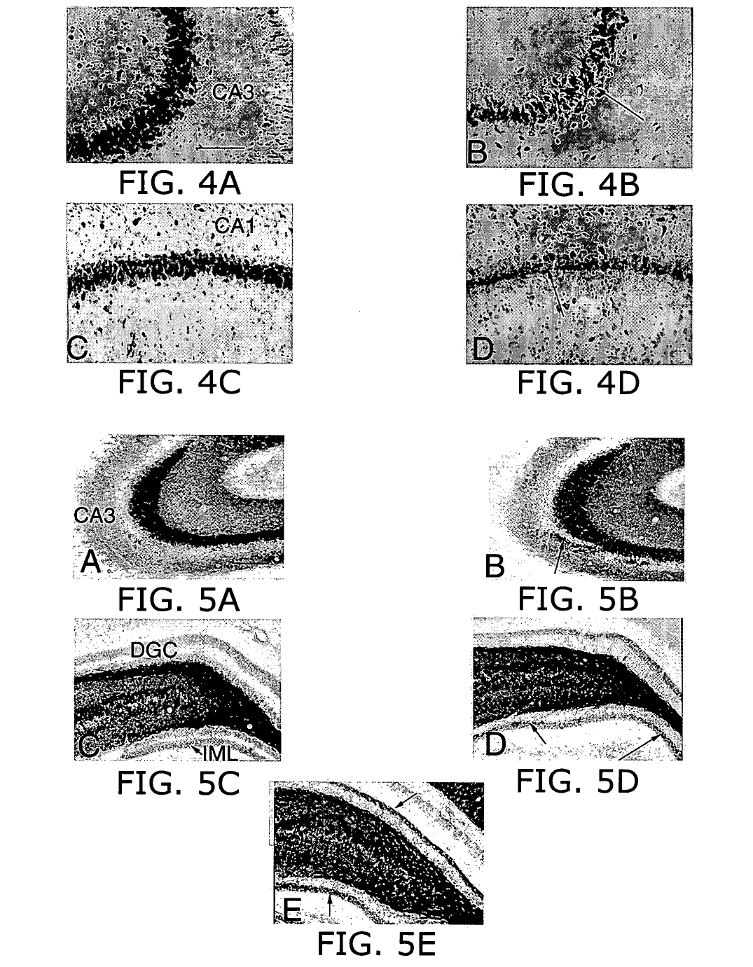

Histological Findings (FIGS. 4 & 5)

The brains of animals from these experimental groups were analyzed as described above for cell loss scores (from thionin stained sections) and sprouting of Timm fibers (from Timm stained sections). Animals receiving pilocarpine-induced status epilepticus had apparent cell loss in CA3, CA1 and the hilus and sprouting of Timm fibers in the supragranular and CA3 hippocampal subfield. As expected, compared to non-seizure rats, animals that had seizures had significantly higher cell loss scores (10.2±3.1 vs. 4.5±2.8, p<0.001) and supragranular Timm scores (1.8±1.0 vs. 0.9±0.7, p=0.03). CA3 Timm scores were also higher in the status epilepticus rats than in the non-seizure rats (1.5±0.7 vs. 0.6±0.4, p<0.01).

The group of animals that received 5 μg or more of encapsulated muscimol prior to pilocarpine (n=9) had significantly less apparent cell loss and Timm staining than the group comprising those that received blank LPSPs or normal saline (n=7). Unpaired Student t-tests revealed that the mean total cell loss score in the encapsulated muscimol group was 4.42±2.38 compared to 11.10±2.37 for rats receiving blank LPSPs and normal saline (p<0.001). The mean Timm stain scores were also significantly lower in the encapsulated muscimol group compared to the blank LPSP and saline group in both the CA3 (0.60±0.50 vs. 1.91±0.65, p<0.001) and supragranular regions (0.82±0.48 vs. 1.62±0.70, p=0.02) of the dentate gyrus.

A further analysis focused on animals that received 5 μg of muscimol and their controls was done to allow comparison between free and encapsulated muscimol (Table 2). Groups of animals receiving 5 μg of either free or encapsulated muscimol had lower total cell loss scores than the groups that receive saline or blank LPSPs. However, there was no significant difference between the protective effects of encapsulated vs. free muscimol. Animals treated with encapsulated muscimol showed less sprouting of Timm fibers in CA3 and supragranular regions than saline-treated animals. In the supragranular region, the Timm score for encapsulated muscimol was lower than for free muscimol. While there was no significant difference between encapsulated and free muscimol in Timm scores in CA3, the score for free muscimol was not lower than that for normal saline (while that for encapsulated muscimol was). Blank LPSPs did not have an effect on total cell loss scores or CA3 Timm scores, but did have lower supragranular Timm scores than in the normal saline group. LPSPs containing muscimol did not have lower Timm scores than blank LPSPs, although the former did have lower scores than saline in CA3 while the latter did not.

TABLE 2
Cell loss and Timm scores according to treatment group

| | Encapsulated | Free | | Normal | |
|---|---|---|---|---|---|
| Outcome | Muscimol | muscimol | LPSP | saline | p-value |
| Total cell loss† | 5.38 ± 3.01 | 6.27 ± 0.57 | 10.02 ± 3.75 | 11.13 ± 1.36 | <0.05^{a} |
| Timm scores | | | | | |
| CA3 region | 0.75 ± 0.70 | 1.48 ± 0.63 | 1.55 ± 0.92 | 2.18 ± 0.24 | <0.05^{b} |
| Supragranular | 0.94 ± 0.43 | 1.75 ± 0.28 | 1.05 ± 0.88 | 1.97 ± 0.11 | <0.05^{c} |

†based on thionin staining.
Data are means ± SD. LPSP = lipid-protein-sugar particles. Dosage was 5 μg in both encapsulated and free muscimol groups.
Sample sizes: for all groups n = 4, except for LPSP where n = 3. All p-values were determined by ANOVA, followed by Fisher's least significant difference procedure for multiple post-hoc comparisons, in which p < 0.05 was considered significant.
^{a}Significantly lower in both encapsulated and free muscimol groups than in both LPSP and normal saline.
^{b}Significantly lower in encapsulated muscimol group than normal saline.
^{c}Significantly lower in encapsulated muscimol compared to free muscimol and normal saline, and LPSP compared to normal saline.

Discussion

Controlled release lipid-protein-sugar particles containing muscimol successfully mitigated the onset of seizures in pilocarpine-treated rats. The effectiveness of muscimol was not adversely affected by the spray-drying manufacture process, nor by coencapsulation with phospholipids, protein, or sugar. On the contrary, the encapsulated formulation showed enhanced anticonvulsant activity compared to the free drug, in terms of both seizure scores and histological injury. This improved performance is unlikely to be due to a separate action of the putatively inert excipients on neurons or glia, or inactivation of pilocarpine by those excipients, since blank particles did not mitigate seizure scores. The latter finding also argues against the possibility that the injected particles—which were placed prior to pilocarpine—somehow acted as a barrier or sponge preventing pilocarpine from inducing seizures.

Muscimol-loaded microparticles also mitigated the histological changes from pilocarpine administration, but were only slightly more effective than free muscimol in doing so. It is possible that this effect will be accentuated in more chronic models of disease, and with formulations that have a more extended timeframe of drug release. The latter is certainly conceivable as microspheres with drug release durations lasting months are available clinically for other indications (Langer R. Drug delivery and targeting. Nature 1998; 392: 5-10; incorporated herein by reference). In this regard it is also encouraging that other investigators, using more macroscopic devices have shown prolonged effectiveness (Kubek A J, Liang D, Byrd K E, Domb A J. Prolonged seizure suppression by a single implantable polymeric-TRH microdisk preparation. Brain Res. 1998; 809: 189-197; Tamargo R J, Rossell L A, Kossoff E H, Tyler B M, Ewend M G, Aryanpur J J. The intracerebral administration of phenytoin using controlled-release polymers reduces experimental seizures in rats. Epilepsy Res 2002; 48: 145-155; each of which is incorporated herein by reference) (see below). Although blank particles did not mitigate seizure activity or cell loss, we cannot exclude the possibility that they had a mild intrinsic protective effect, given their mitigation of supragranular Timm sprouting.

The finding that particles loaded with muscimol prevented clinical seizure activity to a greater extent than free muscimol was, in a way, counter-intuitive. In general, one would expect a given amount of free drug to be more efficacious in the short term than the same amount of drug encapsulated, as it will cause higher drug levels initially. It is possible that the improved efficacy of the encapsulated drug stems from the design of the model employed. The anticonvulsant regimens were administered 80 minutes before the end of the pilocarpine infusion. Free muscimol may have largely diffused away from the site of injection during that interval, while the encapsulated form maintained an effective concentration for a longer time (approximately 80% of the encapsulated drug was released after 80 minutes).

These results demonstrate the potential utility of focally delivered drug-loaded microparticles in the treatment of clinical seizure activity. This is consistent with animals studies showing that a macroscopic implant releasing tetrodotoxin can prevent post-traumatic epileptogenesis (Graber K D, Prince D A. Tetrodotoxin prevents posttraumatic epileptogenesis in rats. Ann. Neurol. 1999; 46: 234-242; incorporated herein by reference), that a polymeric microdisk containing thyrotropin-releasing hormone can suppress kindling expression (Kubek A J, Liang D, Byrd K E, Domb A J. Prolonged seizure suppression by a single implantable polymeric-TRH microdisk preparation. Brain Res. 1998; 809: 189-197; incorporated herein by reference), and that a polymeric device containing phenytoin reduces experimental seizures (Tamargo R J, Rossell L A, Kossoff E H, Tyler B M, Ewend M G, Aryanpur J J. The intracerebral administration of phenytoin using controlled-release polymers reduces experimental seizures in rats. Epilepsy Res 2002; 48: 145-155; incorporated herein by reference). The microparticles described here are individually approximately one hundred times smaller than those devices, and could easily be applied by stereotactic injection through a very fine needle, and, being composed of naturally occurring substances that are both biocompatible and completely biodegradable, would not present a long-term foreign body. It is likely that they would be safe for intracranial use. Similar particles injected into murine cerebral parenchyma did not cause any detectable tissue injury or inflammation. Furthermore, when injected into cerebral ventricles they did not cause obstructive hydrocephalus and, when injected into the internal carotid artery, only had effects on cerebral blood flow when injected rapidly in great quantity (Kohane D S, Plesnila N, Thomas S S, Le D, Langer R S, Moskowitz M A. Lipid-sugar particles for intracranial drug delivery: safety and biocompatibility. Brain Res. 2002; (in press); incorporated herein by reference).

Controlled release of anticonvulsant drugs at the focus of epileptic activity holds several theoretical advantages over systemic delivery by the oral or intravenous routes. Controlled release technology can yield high local concentrations of drug with relatively low total drug release. Only the affected area of the brain will be treated, thereby minimizing neuropsychiatric effects of the drugs. Furthermore, intractable seizure activity treated in this manner might not require the generalized ablation of neural activity that a pentobarbital coma entails, with the associated respiratory depression and hypotension that always necessitate mechanical ventilation and routinely require vasoactive drugs. Microparticles could serve a diagnostic purpose, in helping to demarcate the extent of a seizure focus for eventual ablation. Local controlled release could markedly improve the therapeutic index of the drugs with respect to systemic effects (e.g., hepatotoxicity). Furthermore, since drugs given in this way should achieve much lower systemic levels for a given degree of effectiveness, there should be less induction of hepatic enzymes and other untoward drug interaction.

These particles are attractive vehicles for the delivery of therapeutics because the process by which they are produced—spray drying—is very flexible in terms of drugs and excipients that can be incorporated. Thus, they can be made to contain a range of drugs or drug combinations, allowing for exploration of the local effects of synergistic drug regimens. Similarly the excipients can readily be changed if they are undesirable for some reason (e.g., antigenicity of protein content). Varying the composition of the excipients could also potentially permit modulation of the duration of drug release (Kohane D S, Lipp M, Kinney R, Lotan N, Langer R. Sciatic nerve blockade with lipid-protein-sugar particles containing bupivacaine. Pharm. Res. 2000; 17: 1243-1249; incorporated herein by reference), depending on which of several possible mechanisms (Langer R. Drug delivery and targeting. Nature 1998; 392: 5-10; incorporated herein by reference) are relevant to the release of muscimol and/or other drugs. Such modifications will be important in optimizing the time span over which therapeutic effects can be extended. It also bears mentioning that particles similar to these (Ben-Jebria A, Chen D, Eskew M L, Vanbever R, Langer R, Edwards D A. Large porous particles for sustained protection from carbachol-induced bronchoconstriction in guinea pigs. Pharm. Res. 1999; 16: 555-561; incorporated herein by reference) have been used for inhalational delivery of a variety of compounds. Presumably, therefore, such particles could be used for systemic delivery of anticonvulsant, by inhalation.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A method of treating a neurological disorder, the method comprising steps of:
providing a patient suffering from the neurological disorder or at risk for the neurological disorder;
providing microparticles comprising pharmaceutical agents useful in treating the neurological disorder; and
administering the microparticles into the patient.

2. The method of claim 1, wherein the microparticles are selected from the group consisting of liposomes, spray-dried particles, coacervates, and microspheres.

3. The method of claim 1, wherein the microparticles are less than 1 mm in their largest dimension.

4. The method of claim 1, wherein the microparticles are less than 500 microns in their largest dimension.

5. The method of claim 1, wherein the microparticles are less than 250 microns in their largest dimension.

6. The method of claim 1, wherein the microparticles are less than 100 microns in their largest dimension.

7. The method of claim 1, wherein the pharmaceutical agent is selected from the group consisting of site 1 sodium channel blockers, local anesthetics, glucocorticoid receptor agonists, and anti-epileptic drugs.

8. The method of claim 1, wherein the pharmaceutical agent is selected from the group consisting of site 1 sodium channel blockers, local anesthetics, and glucocorticoid receptor agonists.

9. The method of claim 8, wherein the glucocorticoid receptor agonist is dexamethasone.

10. The method of claim 1, wherein the pharmaceutical agent comprises a site 1 sodium channel blocker, a local anesthetic, and a glucocorticoid receptor agonist.

11. The method of claim 1, wherein the pharmaceutical agent is selected from the group consisting of tetrodotoxin, saxitoxin, neosaxitoxin, decarbamoyl saxitoxin, gonyautoxin, natural and synthetic derviatives of saxitoxin and tetrodotoxin, amino-amide and amino-ester local anesthetics, bupivacaine, lidocaine, tetracaine, dibucaine, vanilloid receptor agonists, capsaicin, resiniferatoxin, hydrocortisone, dexamethasone, phenytoin, benzodiazepines, valproic acid, carbamazepine, felbamate, barbiturates, muscimol, and GABA receptor agonists.

12. The method of claim 1, wherein the step of administering comprises delivering the microparticles to a seizure focus within the brain of the patient.

13. The method of claim 1, wherein the step of administering comprises delivering the microparticles within or onto the brain, cerebrospinal fluid, or cerebral vasculature of the patient.

14. The method of claim 1, wherein the step of administering comprises delivering the microparticles within or onto an iatrogenically created or revealed site, plane, or cavity within the brain.

15. The method of claim 1, wherein the microparticles comprise a targeting agent.

16. The method of claim 15, wherein the targeting agent is selected from the group consisting of antibodies, fragments of antibodies, low-density lipoproteins (LDLs), transferrin, asialycoproteins, gp120 envelope protein of the human immunodeficiency virus (HIV), carbohydrates, receptor ligands, TAT sequence, and sialic acid.

17. The method of claim 1, wherein the microparticles are triggered to release the agent via radio-frequency beams, infrared, magnetism, osmotic changes, pH changes, electrical activity, or the presence of a particular triggering agent.

18. The method of claim 1 comprising additional step of compressing, complexing, or cross-linking the microparticles to form a macroscopic pellet prior to delivery.

19. The method of claim 1, wherein the neurological disorder is epilepsy.

20. The method of claim 1, wherein the neurological disorder is selected from the group consisting of Parkinson's disease, Huntington's disease, Alzheimer's disease, tremor, hemiballismus, and choreas.

21. A method of treating cardiac arrhythmia, the method comprising steps of:
providing a patient suffering from a cardiac arrhythmia or at risk of a cardiac arrhythmia;
providing microparticles comprising at least one pharmaceutical agent useful in the treatment of arrhythmias; and
administering the microparticles to an arrhythmic focus of the heart of the patient.

22. The method of claim 21, wherein the pharmaceutical agent is selected from the group consisting of phenytoin, lidocaine, adrenergic agonists, dopamine, bromocriptine, pergolide, pramipexole, ropirinole, anticholinergic agents, benztropine, trihexyphenyydyl, biperyden, monoamine oxidase inhibitors, carbidopa, COMT inhibitors, GABA receptor agonists, benzodiazepines, muscimol, clozapine, risperisdone, zyprexa, tricyclic antidepressants, serotonin reuptake inhibitors, antipsychotic drugs, adrenergic antagonists, amiodarone, and procainamide.

23. The method of claim 21, wherein the pharmaceutical agent is selected from the group of Class I, II, III, IV, and V antiarrhythmic drugs.

24. The method of claim 21, wherein the pharmaceutical agent is selected from the group consisting of quinidine, procainamide, disopyramide, lidocaine, mexiletine, tocainide, phenytoin, flecainide, encainide, propafenone, propanolol, nadolol, pindolol, labetablol, timolol, metoprolol, acebutolol, atenolol, esmolol, alprenolol, bretylium, amiodarone, sotalol, verapamil, and dilitiazem.

25. The method of claim 21, wherein the pharmaceutical agent is selected from the group consisting of site 1 sodium channel blockers, local anesthetics, and glucocorticoid receptor agonists.

26. The method of claim 21, wherein the pharmaceutical agent comprises a site 1 sodium channel blocker, a local anesthetics, and a glucocorticoid receptor agonist.

27. The method of claim 26, wherein the glucocorticoid receptor agonist is dexamethasone.

28. The method of treating preterm labor, the method comprising steps of:
providing a patient suffering from preterm labor or at risk for preterm labor;
providing microparticles comprising at least one tocolytic agent useful in stopping or preventing tocolysis; and
administering the microparticles nearby or to the uterus of the patient.

29. The method of claim 28, wherein the tocolytic agent is selected from the group consisting of adrenergic agonists and calcium channel blockers.

30. The method of claim 28, wherein the pharmaceutical agent is selected from the group consisting of site 1 sodium channel blockers, local anesthetics, and glucocorticoid receptor agonists.

31. The method of claim 28, wherein the pharmaceutical agent comprises a site 1 sodium channel blockers, local anesthetics, and a glucocorticoid receptor agonist.

32. The method of claim 28, wherein the pharmaceutical agent comprises a site 1 sodium channel blocker.

33. The method of claim 28, wherein the pharmaceutical agent comprises a site 1 sodium channel blocker and another agent selected from the group consisting of calcium channel blockers, adrenergic agonists, local anesthetics, and glucocorticoid receptor agonists.

34. The method of claim 28, wherein the pharmaceutical agent comprises a site 1 sodium channel blocker and another agent selected from the group consisting of local anesthetics and glucocorticoid receptor agonists.

35. A pharmaceutical composition for suppressing electrical activity in an electrically excitable tissue comprising a site 1 sodium channel blocker, a local anesthetic, and a glucocorticoid receptor agonist.

36. The composition of claim 35, wherein the tissue is brain.

37. The composition of claim 35, wherein the tissue is heart.

38. The composition of claim 35, wherein the tissue is uterus.

39. The composition of claim 35, wherein the site 1 sodium channel blocker is selected from the group consisting of tetrodotoxin, saxitoxin, neosaxitoxin, decarbamoyl saxitoxin, gonyautoxin, and derivative thereof.

40. The composition of claim 35, wherein the local anesthetic is selected from the group consisting of benzocaine, bupivacaine, cocaine, etidocaine, lidocaine, mepivacaine, pramoxine, prilocaine, procaine, procainamide, proparacaine, ropicaine, tetracaine, and dibucaine.

41. The composition of claim 35, wherein the glucocorticoid receptor agonist is selected from the group consisting of hydrocortisone, dexamethasone, cortisone, prednisone, beclomethasone, betamethasone, flunisolide, methyl prednisone, paramethasone, prednisolone, triamcinolome, alclometasone, ancinonide, clobetasel, fluorocortisone, diflurosone diacetate, flucinolone acetonide, fluoromethalone, flurandrenolide, halcinonide, medrysone, and mometasone.

42. The composition of claim 35, wherein the glucocorticoid receptor agonist is dexamethasone.

43. A pharmaceutical composition for suppressing electrical activity in an electrically excitable tissue comprising a site 1 sodium channel blocker, and another agent selected from the group consisting of local anesthetics and glucocorticoid receptor agonists.
